(19) United States
(12) Patent Application Publication
(10) Pub. No.: US 2008/0159966 A1
(43) Pub. Date: Jul. 3, 2008

(54) Multiphase Toothpaste Composition

(76) Inventors: Rupesh Kumar Agarwal, Mumbai (IN); Ilaria Faravelli, Milano (IT); Donald Peter Gregory, Wirral (GB); Brian Joseph Groves, Wirral (GB); Philip Christopher Waterfield, Wirral (GB)

Correspondence Address: UNILEVER PATENT GROUP, 800 SYLVAN AVENUE, AG West S. Wing, ENGLEWOOD CLIFFS, NJ, 07632-3100, US

(21) Appl. No.: 11/883,674

(22) PCT Filed: Jan. 6, 2006
(86) PCT No.: PCT/EP06/00114

§ 371 Date: Aug. 3, 2007

(30) Foreign Application Priority Data
Feb. 4, 2005 (IN) 0118/MUM/2005
Apr. 8, 2005 (IN) 0291/KOL/2005

(51) Int. Cl. A61K 8/02; A61Q 11/00

(52) U.S. Cl. 424/49

(57) ABSTRACT

Multiphase toothpaste composition comprising a first phase disposed co-axially within a second phase, each of the phases having a viscosity of from 180 000 to 250 000 mPa·s.

The present invention relates to a multiphase toothpaste composition.

WO 99/01342 discloses apparatus for inserting plural materials into containers. The apparatus comprises a nozzle with a first hollow member and a second hollow member arranged inside the first hollow member. The nozzle is designed for directing the extrusion of multiple toothpaste phases into a toothpaste container with one phase being arranged within another.

Despite the prior art there remains the need for multiphase toothpaste compositions with improved visual impact and improved, more consistent dispensing from a tube.

The present inventors have found that improved visual impact and better, more consistent dispensing from a tube is achieved by raising and carefully configuring the viscosities of each phase. Accordingly and in a first aspect, the present invention provides a multiphase toothpaste according to claim 1.

The first phase is disposed co-axially within the second phase. By this is meant that the longitudinal axis of the ribbon as dispensed from the toothpaste container falls within the inner, first phase. Similarly, the general, longitudinal axis of the toothpaste as stored within the toothpaste container falls within the inner, first phase.

Such alignment is understood to be judged by the eye and not mathematically.

When looking at a section of the dispensed ribbon end-on the inner phase may extend towards or up to the exterior surface of the ribbon. Such extension may be radial, spiral or abstract and, where it is radial or spiral it may be regular or irregular. Regular radial extensions are the most preferred to the consumer. In addition the core may, in cross section be of any shape, for example, star-shaped, square shaped, triangular, etc. These shaped cores are a real plus to the younger consumers. However, it is most preferred that the inner phase comprises no extensions and instead provides nothing more than a regular core to the ribbon. This regular shape is less fussy and provide clean lines to the product which reinforces the impression of cleanliness that toothpastes aim to provide.

The phases according to the composition of the invention have viscosities as measured on a Brookfield RV DV-1 viscometer at 25° C. and 5 rpm using a TD spindle from 180 000 mPa·s to 250 000 mPa·s, preferably from 200 000 to 240 000 mPa·s, more preferably from 210 000 to 230 000 mPa·s and most preferably from 210 000 to 225 000 mPa·s. Such viscosities provide the best performance with regard to extrusion into the container and also from the container by the consumer. These viscosity ranges provide the best extrusion performance for coaxially arranged phases as described herein. The extruded paste is stable and of consistent width on dispensing onto a toothbrush.

Preferably, the outer, second phase is a visually clear gel. By visually clear is meant that the inner, first phase can be seen through the outer, second phase. Having two visually clear phases also provides an attractive composition for the consumer and is a preferred embodiment of this invention.

In a preferred embodiment the first phase constitutes up to 25% of the volume of the toothpaste composition. Should the inner phase constitute much more than 25% by volume of the composition the inner phase often appears to overwhelm the composition such that the outer phase is hardly noticed at all by the consumer. This is particularly the case when the outer phase is visually clear.

Preferably, the inner first phase constitutes from 11 to 20% and more preferably from 13 to 18% by volume of the toothpaste composition.

The first and second phases may be the same or different with regard to their principle components, i.e. thickeners, actives, structurants and abrasives. Where the first and second phases are essentially the same they may differ in minor components such as colours or flavours.

In an alternative preferred embodiment the inner, first phase is opaque. This adds a further benefit in that it creates a silvery effect at the interface between the two phases. This is especially so when the outer phase is visually clear and is an attractive effect for the consumer.

In an alternative preferred embodiment at least one of the phases is coloured. Preferably, the outer phase is coloured. This is even more preferred when the outer phase is visually clear. Preferred colours include green, red, orange, yellow, blue, gold and purple.

In a further preferred embodiment either of the phases, especially when a visually clear phase is required, comprises abrasive silica. The particular abrasive silica used in the present invention is a silica with a low refractive index. It may be used as the sole abrasive silica, or in conjunction with a low level of other abrasive silicas, e.g. those according to EP 236 070. The low refractive index silicas, used as abrasives in the present invention are preferably silicas with an apparent refractive index (R.I.) in the range of 1.41-1.47, preferably 1.435-1.445, preferably having a weight mean particle size of between 5 and 15 mm, a BET (nitrogen) surface area of between 10 and 100 m²/g and an oil absorption of about 70-150 cm³/100 g, but abrasive silicas with a lower apparent refractive index may also be used. Typical examples of suitable low refractive index abrasive silicas (e.g. having an R.I. of between 1.435 and 1.445) are Tixosil 63 and 73 ex Rhone Poulenc; Sident 10 ex Degussa; Zeodent 113 ex Zeofinn; Zeodent 124 ex Huber, Sorbosil AC 77 ex Crosfield Chemicals (having an R.I. of approximately 1.440). The amount of these silicas in the composition generally ranges from 5-60% by weight, usually 5-20% by weight.

In a preferred embodiment the inner phase comprises chalk, preferably fine ground natural chalk. The inner composition will preferably comprise chalk at from 10 to 60% by weight of the phase.

The phases of the composition according to the invention are manufactured using standard processes. They may be extruded into a container for dispensing by equipment such as that discussed in detail in WO 99/01342, i.e. a coaxial nozzle assembly attached to standard equipment.

The toothpaste composition according to the present invention can comprise an agent selected from the group consisting of anti-carries agents, tooth whitening agents, anti-tartar agents, anti-malodour agents, anti-gingivitis agents and mixtures thereof.

The toothpaste composition will comprise further ingredients which are common in the art, such as:

antimicrobial agents, e.g. chlorhexidine, sanguinarine extract, metronidazole, quaternary ammonium compounds, such as cetylpyridinium chloride; bis-guanides, such as chlorhexidine digluconate, hexetidine, octenidine, alexidine; and halogenated bisphenolic compounds, such as 2,2′ methylenebis-(4-chloro-6-bromophenol);

anti-inflammatory agents such as ibuprofen, flurbiprofen, aspirin, indomethacin etc.;

anti-caries agents such as sodium- and stannous fluoride, aminefluorides, sodium monofluorophosphate, sodium trimeta phosphate and casein; plaque buffers such as urea, calcium lactate, calcium glycerophosphate and strontium polyacrylates;

vitamins such as Vitamins A, C and E;

plant extracts;

desensitizing agents, e.g. potassium citrate, potassium chloride, potassium tartrate, potassium bicarbonate, potassium oxalate, potassium nitrate and strontium salts;

anti-calculus agents, e.g. alkali-metal pyrophosphates, hypophosphite-containing polymers, organic phosphonates and phosphocitrates etc.;

biomolecules, e.g. bacteriocins, antibodies, enzymes, etc.;

flavours, e.g. peppermint and spearmint oils;

proteinaceous materials such as collagen;

preservatives;

opacifying agents;

colouring agents;

pH-adjusting agents;

sweetening agents;

pharmaceutically acceptable carriers, e.g. starch, sucrose, water or water/alcohol systems etc.;

surfactants, such as anionic, nonionic, cationic and zwitterionic or amphoteric surfactants;

particulate abrasive materials such as silicas, aluminas, calcium carbonates, dicalciumphosphates, calcium pyrophosphates, hydroxyapatites, trimetaphosphates, insoluble hexametaphosphates and so on, including agglomerated particulate abrasive materials, usually in amounts between 3 and 60% by weight of the oral care composition. Preferred abrasives are chalk and silica, more preferably fine ground natural chalk.

Humectants such as glycerol, sorbitol, propyleneglycol, xylitol, lactitol etc.;

binders and thickeners such as sodium carboxymethyl-cellulose, hydroxyethyl cellulose (Natrosol®), xanthan gum, gum arabic etc. as well as synthetic polymers such as polyacrylates and carboxyvinyl polymers such as Carbopol®;

polymeric compounds which can enhance the delivery of active ingredients such as antimicrobial agents can also be included;

buffers and salts to buffer the pH and ionic strength of the oral care composition; and

other optional ingredients that may be included are e.g. bleaching agents such as peroxy compounds e.g. potassium peroxydiphosphate, effervescing systems such as sodium bicarbonate/citric acid systems, colour change systems, and so on.

Liposomes may also be used to improve delivery or stability of active ingredients.

In a second aspect the present invention provides a toothpaste container comprising a first and second phase as described in the first aspect of the invention stored within a tubular container, the container comprising a tubular body which is crimped at one end and comprises a dispensing assembly at the other through which the composition is extruded by the consumer.

Preferably, the tubular container is transparent or translucent so that the inner phase can be seen within the outer phase within the container.

An embodiment of the invention is now discussed in the non-limiting example.

### EXAMPLES

This composition comprises a first phase and a second phase. The second phase is visually clear and the inner phase is opaque. The composition comprises 85% v/v of the first phase and 15% v/v of the second phase.

| | % (w/w) of the | % (w/w) of the |
|---|---|---|
| Ingredient | first phase | second phase |
| water | 12.73 | 12.48 |
| sorbitol (70% aq) | 63.00 | 63.00 |
| sodium fluoride | 0.32 | 0.32 |
| polyethylene glycol | 4.00 | 4.00 |
| (PEG 32) | | |
| titanium dioxide | 0.00 | 0.50 |
| thickening silica | 9.25 | 9.00 |
| Abrasive silica | 8.00 | 8.00 |
| sodium | 0.90 | 0.90 |
| carboxymethylcellulose | | |
| sodium lauryl sulphate | 1.80 | 1.80 |
| flavours and colours | trace | trace |

1. Multiphase toothpaste composition comprising a first phase disposed co-axially within a second phase, each of the phases having a viscosity of from 180 000 to 250 000 mPa·s.

2. Multiphase toothpaste composition according to claim 1 comprising a first phase disposed co-axially within a second phase, each of the phases having a viscosity of from 200 000 to 240 000 mPa·s.

3. Multiphase toothpaste composition according to claim 1 comprising a first phase disposed co-axially within a second phase, each of the phases having a viscosity of from 210 000 to 230 000 mPa·s.

4. Multiphase toothpaste composition according to claim 1 comprising a first phase disposed co-axially within a second phase, each of the phases having a viscosity of from 210 000 to 225 000 mPa·s.

5. Multiphase toothpaste composition according to claim 1 wherein the second phase is a visually clear gel phase.

6. Multiphase toothpaste composition according to claim 1 wherein the first phase is opaque.

7. Multiphase toothpaste composition according to claim 1 wherein the first phase constitutes up to 25% by volume of the toothpaste composition.

8. Multiphase toothpaste according to claim 1 wherein the first phase constitutes from 11 to 20% by volume of the toothpaste composition.

9. Multiphase toothpaste composition according to claim 1 wherein the first phase constitutes from 13 to 18% by volume of the toothpaste composition.

10. Multiphase toothpaste composition according to claim 1 wherein at least one of the phases is coloured.

11. Multiphase toothpaste composition according to claim 1 wherein the visually clear gel phase comprises abrasive silica.

12. Multiphase toothpaste composition according to claim 1 wherein the first phase comprises chalk as abrasive.

13. Multiphase toothpaste composition according to claim 12 wherein the chalk is fine ground natural chalk.

14. Multiphase toothpaste composition according to claim 1 wherein the second phase comprises an abrasive silica having a Refractive Index of from 1.41 to 1.47.
